(19) United States
(12) Patent Application Publication
(10) Pub. No.: US 2020/0384140 A1
(43) Pub. Date: Dec. 10, 2020

(54) UV DISINFECTION APPARATUS, METHOD THEREFOR, AND USE THEREOF

(71) Applicant: Q One Holding AG, Sarnen (CH)

(72) Inventor: Bernd Hoehne, Achim (DE)

(21) Appl. No.: 16/955,163

(22) PCT Filed: Dec. 20, 2017
(86) PCT No.: PCT/DE2017/101093

§ 371 Date: Jun. 18, 2020

(51) Int. Cl. A61L 2/10; D06F 35/00

(52) U.S. Cl. CPC A61L 2/10; D06F 35/008; A61L 2202/122; A61L 2202/26; A61L 2202/14; A61L 2202/11; A61L 2202/17

(57) ABSTRACT

A UV disinfection apparatus (1, 1′), with a housing (10) and a UV light source (11) therein. An electrical energy store (15) is provided in the housing (10). The housing (10) is sealed off, and a sensor is provided therein, which activates the UV light source (11) only when the laundry treatment device (2) is in operation. Also, a method for disinfecting laundry accommodated in a rotatable drum (21) by means of a UV disinfection apparatus (1, 1′) which is autonomous placed into or set in the drum (21) together with the laundry. The UV light source (11) is only activated when the laundry treatment device is in operation.

The invention relates to a UV disinfection apparatus with a housing and a UV light source accommodated therein. Furthermore, the invention relates to a method for disinfecting laundry accommodated in a rotatable drum of a laundry treatment device by means of a UV disinfection apparatus as well as a use for it.

Laundry treatment machines are known for the treatment, namely cleaning, washing and drying of laundry in various configurations for private use in the household as well as professional uses. Laundry treatment machines are in particular washing machines, washer-dryers and dryers. All these machines have in common that the laundry to be treated is taken up in a receptacle and the receptacle is moved to mix and treat the laundry. The receptacle is designed as a rotatable drum. Water is used for washing, if necessary with the addition of detergents as lye or with chemical additives or air, which is heated, is used for drying. During the circulation or after the washing process is completed, the circulated fluid is released into the environment or into the drain line. For a further treatment process, fresh water or, in the case of a drier, fresh air is added. However, germs may still be present in the water or air discharge and, of course, also in the treated laundry, which may still be active after the laundry treatment, depending on the washing treatment temperature and ingredients, and may even multiply particularly well in the environment. Accordingly, the fluid to be discharged, which is no longer required, is possibly contaminated with a considerable number of bacteria. Furthermore, the items of laundry can still carry a considerable number of germs after removal from the laundry treatment machine. All of this is undesirable for hygienic reasons.

From EP 2 314 753 A1 a washing process is known, in which laundry in a washing machine is flushed with water or wash liquor and in the recirculation of the rinse water this rinse water is disinfected with UV irradiation.

A similar washing machine is known from EP 1 922 443 B1, in which the washing water in a sump area is irradiated with UV light, wherein shields are provided, to prevent an undesired irradiation of UV sensitive objects.

EP 2 236 660 A1 describes a washing machine in which a UV irradiation device is installed in the region of the drum, wherein the irradiation is carried out during rotation of the drum.

U.S. Pat. No. 6,877,248 B1 discloses a clothes dryer, wherein UV light sources are provided outside the rotatably driven drum for disinfecting the laundry contained therein.

Therefore, various types of washing machines and driers are known provided with special UV irradiation units that allow disinfection of the laundry treated therein. However, this requires specialized customized laundry treatment appliances so that a retrofitting of older machines or, if necessary, supplementation of this UV disinfection in conventional laundry appliances is not possible.

The object of the invention is, therefore, to enable a UV disinfection apparatus or treatment in conventional laundry treatment machines in simplest possible manner.

This object is achieved with a UV disinfection apparatus according to claim 1, a disinfection process according to claim 10 and a use of the UV disinfection apparatus according to claim 14.

By providing an electrical energy store or accumulator in the housing, sealing the housing and providing a sensor therein, which activates the UV light source only during operation of the laundry treatment device, the disinfection apparatus can be used in the drum independent of the laundry treatment apparatus. The electrical energy store, in particular a rechargeable battery, serves to supply the disinfection apparatus with electrical energy. The sealed casing prevents wash water or lye or other fluids penetrating into the UV disinfection unit, wherein a sensor in the device ensures that the UV irradiation can not be activated outside of the laundry treatment device, in particular can be activated only during operation of the laundry treatment apparatus. Thus, the operator is protected from possible unwanted UV exposure.

Accordingly, the UV disinfection apparatus which is independent from the laundry treatment machine is inserted or laid with the laundry in the drum, wherein the UV light source in the UV disinfection unit is switched on only upon operation of the laundry treatment machine, so that the UV disinfection apparatus is used for disinfecting laundry in a washing machine or is used in a dryer.

In a further embodiment fastening means are provided, with which the UV disinfection unit is mounted on the inner side of a drum door of the laundry treatment apparatus. Alternatively, the UV disinfector can also be arranged on the outside of the drum door, provided that the glass drum door has sufficient UV light transmittance. As fastening means, a magnetic attachment or attachment by means of adhesive as well as a stapled/clamp connection can be provided to the UV disinfection unit to be mounted on the inside or outside of the drum door. The UV light source in this embodiment of the UV disinfection apparatus is oriented so as to radiate into the interior of the drum. By covering the drum door by the device arranged there, an additional obscuration from the outside may be omitted even in the case of a glass drum door, to the extent a sufficient shielding of the UV radiation can be carried out with this arrangement.

Alternatively, the UV disinfection unit is formed as a freely movable in the drum, self-sustaining, three-dimensional object, in particular in the form of a ball. In this embodiment, the UV disinfection unit can easily be introduced into the drum of the laundry treatment machine with the laundry to be treated, wherein during operation of the laundry treatment machine the rotating drum ensures complete mixing, and thus exposure to UV radiation, of the totality of the laundry and the washing water located in the drum.

When a position, movement and/or temperature sensor is provided in the housing, it can be determined whether the laundry treatment device is in operation or the UV disinfector is inside the drum by a location determination and/or motion detection and/or temperature detection in the UV disinfection device. In this case all three sensors may be included, so that for safety reasons an activation of the UV light source is possible only upon detection of a location within the drum and the corresponding movement of the drum as well as an elevated washing temperature compared to room temperature, e.g. 30° C., 40° C. or 60° C. Thus it can be safely ruled out that the UV disinfection unit begins radiation with the UV before closing and turning on the laundry treatment apparatus.

Similarly, the end of the disinfection process can be detected on the basis of cessation of motion (stationary drum) and thus the UV light source is turned off.

In order to measure the quality of disinfection, germ sensors are arranged in the UV disinfection apparatus. Thus the quality of disinfection in the fluid circulation path in the laundry treatment machine may be measured and dependent thereon the intensity and/or duration of the UV irradiation be regulated.

If a motion profile is stored in the sensor for detecting the rotational movement of the drum, a false activation of the UV light irradiation upon an unintended handling or moving the UV disinfection apparatus outside the laundry treatment machine can be avoided. The UV light source is activated only when the exact stored motion profile is detected by the sensor.

To ensure a uniform as possible UV exposure of the laundry placed in the laundry treatment apparatus, the UV light source in the housing comprises multiple spatially distributed, for example three to six, UVC emitting LED semiconductors, wherein these are provide in the housing with UV light transparent partition walls. The UV light-permeable partition wall inset in the housing allows an intensive UV light irradiation and at the same time a hermetically sealed enclosure.

If the UV disinfection apparatus is associated with a charging unit for inductive charging of the electric energy storage device, wherein the UV disinfection apparatus can after use be placed on it, the UV disinfection unit can be inductively charged via the charging unit.

In order to avoid an undesirable adverse effect on the environment around the drum door, in particular for glass drum doors, due to UV light emission to the environment, the UV disinfection unit is associated with a cover, wherein the cover is externally fittable into the drum door. The cover can, for example, be clamped or glued to the drum door on the outside.

If the UV disinfection unit is assigned complementary position measuring points, which are attachable to the laundry treatment device, and which cooperate with the position sensor according to claim 4, it can reliably be determined from the UV disinfection apparatus, whether it is located inside of the drum or not. If the position measuring points are attached to the cover for the drum door, it can at the same time be avoided that the UV disinfection apparatus can be switched on if the cover is forgotten.

In addition, the UV disinfector may include thermal insulation to protect the electronics incorporated in the device from excessive heat or cold. The device must be designed in such a way that it can withstand typical washing temperatures of up to 60° C. over the duration of one washing cycle, for example 90 minutes. Additional disinfection is not necessary at higher washing temperatures, as any bacteria and viruses are killed by the high washing temperature. Accordingly the main application is washing at temperatures up to 60° C., and especially in the washing cycles with 30° or 40° C. Optionally, in addition to protection against excessive temperatures or for switching off the UV irradiation, a temperature sensor may be included in the UV disinfector so that for higher wash temperatures an unnecessary disinfection is omitted by non-activation of the UV light source.

Further, the UV disinfector can also be controlled remotely, in order to be able to turn on or off appropriate disinfection programs from the outside. The signal can be transmitted, for example, via known radio connections, such as Bluetooth or WLAN or the like.

Two exemplary embodiments of the invention are described in detail below with reference to the accompanying drawings.

There is shown in:

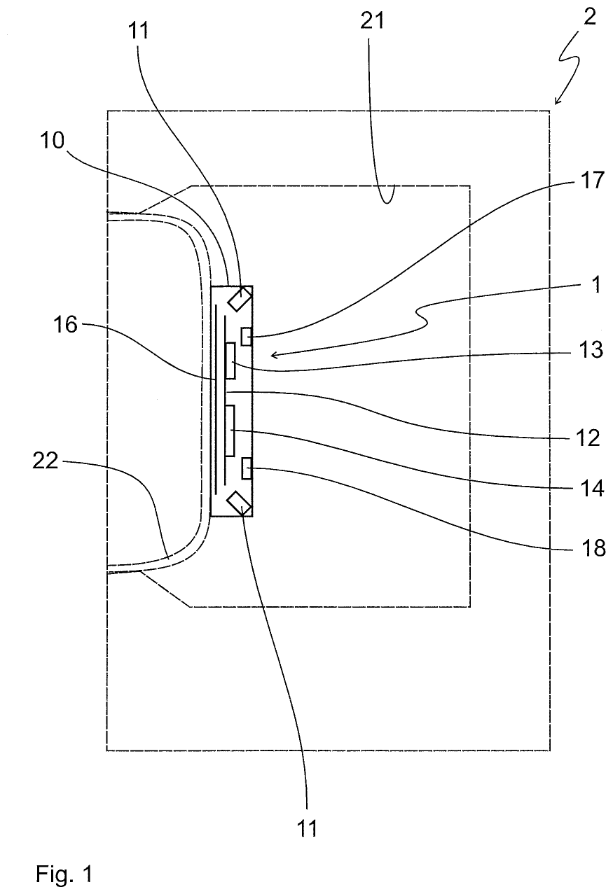

FIG. 1 a UV disinfection unit in a first embodiment in a partly sectioned side view, mounted in a washing machine,

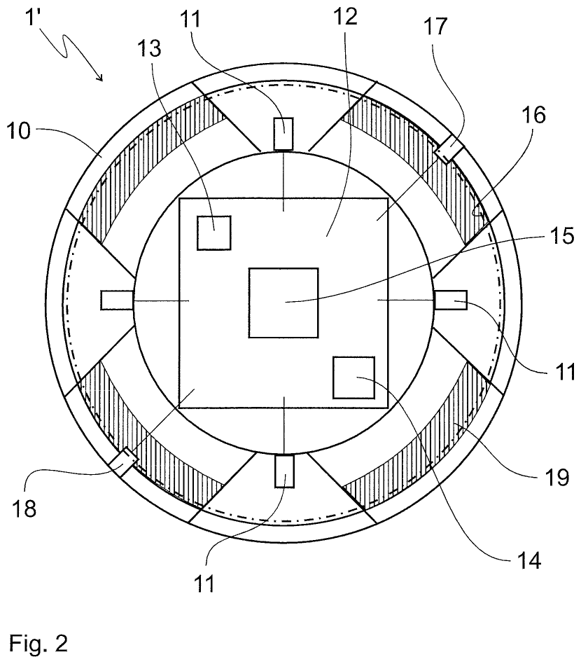

FIG. 2 a UV disinfection unit in a second embodiment in ball shape in cross-section, and

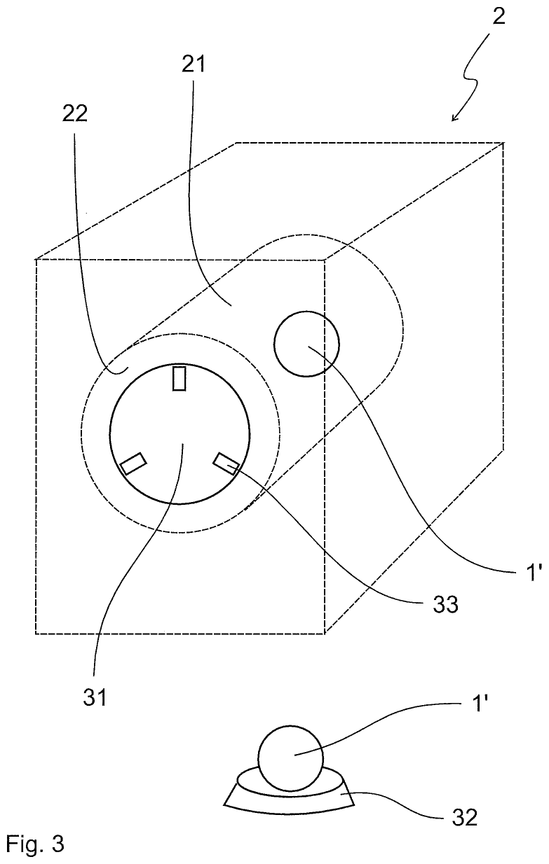

FIG. 3 a diagrammatic perspective view of a UV disinfection apparatus according to FIG. 2 in a washing machine drum.

In FIG. 1, a UV disinfection apparatus 1 is shown with a waterproof housing 10 in a first embodiment in a partly sectioned side view of a washing machine 2, wherein said UV disinfector 1 is attached at the inner side of a drum door of the laundry treatment device 2, for example, a washing machine.

In a second embodiment, the UV disinfector 1′ has a housing 10 in spherical or ball shape. This second embodiment of the UV disinfector 1′ includes in principle the same components as the first embodiment within the housing 10, such as the UV disinfection apparatus 1. Hereinafter, the components of the UV disinfector 1, 1′ is described with reference to the second embodiment, as shown in FIG. 2 in cross-section.

In the fluid-tight housing 10 of the UV disinfection apparatus 1′ there are disposed in the spherical shape as UV light source 11 six UVC-emitting LED-semiconductors, of which four LEDs are visible in the cross section shown in FIG. 2. Two further UVC-emitting LEDs point forwards and backwards perpendicular to the plane of the drawing and are not shown in the cross-sectional representation in FIG. 2. Furthermore, a circuit board 12 with electronic components mounted thereon is provided in the housing 10. Among other things, a position sensor 13 and a movement sensor 14 are provided on the circuit board 12. Furthermore, a germ sensor 17 arranged on the outside of the housing 10, a temperature sensor 18 likewise arranged on the outside, and an electrical energy store 15 in the form of a rechargeable accumulator are connected to the circuit board 12. The housing 10 has insulation 19.

Further, on the back of the UV disinfector 1 according to the first embodiment a charging coil 16 is provided, which allows the inductive charging of the electric energy storage device 15 within the UV disinfection apparatus 1, as shown in FIG. 1 in the cross-sectional representation. For charging the accumulator 15, a correspondingly adapted charging device or charging shell 32 can be provided on the outside of the drum door 22 of the washing machine 2 according to the first illustrative embodiment, so that inductive charging of the accumulator 15 via the charging device 32 and charging coil 16 is made possible. The charging device 32 is, for example, clamped or fastened to the drum door 22 magnetically or by adhesion. To shield the UV light radiation acting in the drum 21, a cover 31 is clamped on the outside of the drum door 22 in such a manner that accidental dropping of the cover 31 is not possible.

In order to determine the position of the UV disinfector 1, 1′ within the drum 21 of the laundry treatment apparatus 2 with the position sensor 13, position reference 33 can, for example, be arranged around the drum 21 so that the position of the UV disinfector 1, 1′ can be determined with certainty. Suitable as a position reference 33 is for example a RFID transponder. In the embodiment shown in FIG. 3 three position sensors 33 are arranged on the cover 31 of the drum door 22 of the washing machine 2 so that they provide active feedback at the prompting of the position sensor 13, so that the actual position of the UV disinfector 1, 1′ can be determined. Therewith it is also ensured that the UV disinfector 1, 1′ can not be turned on in the absence of cover 31 and thus a not possible location determination due to the absence of the position transducer 33 protects the operator against harmful UV irradiation.

Furthermore, memory modules are provided on the circuit board 12, which contain a movement profile or different movement profiles of a washing machine for comparison with the movement of the disinfection apparatus 1′ actually measured with the movement sensor 14 in the second embodiment.

Hereinafter, the use and working of the UV disinfector 1, 1′ is described in particular with reference to the second embodiment of the UV disinfector 1′. The ball-shaped UV disinfection unit 1′ in its idle state is located on a charging cradle 32. This has an excitation coil which cooperates with the charging coil 16 provided in the housing 10 of the UV disinfector 1′ for inductive charging of the accumulator 15 in the housing 10 of the UV disinfector 1′.

If the UV disinfector 1′ is taken from the charger 32 the positional 13, the electronics on the circuit board 12 are changed to an activated state, at which the temperature 18 and/or movement sensors 14 are activated, in which however, the UV light source 11 is still is not activated. The ball-shaped disinfection apparatus 1′ is now introduced into the drum 21 of the washing machine 2 together with the laundry to be treated. After closing the drum door 22, the RFID position transducer 33 located on the cover 31 attached to the drum door 22 signal to the position sensor 13 of the UV disinfector 1′ in the housing 10 that this is positioned within the drum 21.

However, the electronics on the circuit board 12 does not switch on the UV light source 11 in the form of four UVC emitting LED semiconductors, because the drum 21 does not rotate. As soon as the washing machine 2 is switched on and the drum 21 rotates, the movement sensor 14 registers corresponding movement signals which are compared in the electronics of the circuit board 12 with a movement profile stored there in memory. Once the typical motion profile of a rotating drum 21 of a washing machine 2 is detected, the UV light sources 11 are activated, and the laundry 21 located in the drum is disinfected by action of the UV irradiation.

Additionally or alternatively, the activation of the UV light source 11 can be made dependent on the ambient temperature measured with the temperature sensor 18 in the drum 21. For example, at least 30° C. must be on hand at the temperature sensor 18.

The duration of the UV irradiation depends on pre-set irradiation times of a few minutes, for example 1 to 90 minutes, and/or the termination of the rotational movement, namely the end of the selected washing program determined by the motion sensor 14, as a result of which the UV radiation is stopped.

Alternatively or additionally, a germ sensor 17 may be located in the UV disinfector 1, 1′, which measures the number of bacteria in the environment around the UV disinfector 1, 1′ and thus can determine the quality of the performed UV disinfection. With a sufficient reduction in the number of bacteria the UV disinfection can be concluded.

Overall, the sensors, in particular, the position sensor 13, the motion sensor 14 and/or the temperature sensor 18, provide that the UV light source 11 in the disinfecting device 1, 1′ is only turned on when washing or drying is in progress, namely the disinfection apparatus 1, 1′ is located within the drum 21, and the drum 21 rotates or an elevated temperature is detected.

An advantage of the self-sufficient UV disinfection apparatus 1, 1′ according to the two described embodiments is that they can be used in any laundry treatment devices, such as washing machine, washer-dryer or dryer, without the washing treatment devices having to be laboriously rebuilt. Depending on the design of the laundry treatment device 2, in the case of transparent drum doors 22 (glass doors), the cover 31 on the outside of the drum door 22 or on the inside is necessary or actually already provided by mounting the UV disinfection apparatus 1 on the inside. If a position determination is desired with a position sensor 13 and corresponding position references 33, it is essentially only necessary to provide three RFID transponders, for example, glued to the outside of the drum door 22 or to the cover 31. Since the rechargeable battery is recharged inductively, no plug connections are required to be accommodated in the washing drum 21 which could possibly endanger the water tightness of the disinfection apparatus 1, 1′.

### REFERENCE SYMBOL LIST

1, 1′ UV disinfection apparatus

10 housing

11 UV light source, UVC-emitting LED semiconductors

12 circuit board

13 position sensor

14 motion sensor

15 electrical energy storage, accumulator

16 charging coil

17 germ sensor

18 temperature sensor

19 insulation

2 laundry treatment device, washing machine, dryer, washer-dryer

21 drum

22 drum door

31 cover

32 charger, charging cradle

33 position reference

1. A UV disinfection unit (1, 1′) having a housing (10) and a UV light source (11) received in the housing (10), wherein an electrical energy store (15) is provided in the housing (10), the housing is sealed (10) and a sensor is provided in the housing (10), which sensor activates the UV light source (11) only during operation of a laundry treatment apparatus (2).

2. The UV disinfection apparatus (1) according to claim 1, wherein fastening means are provided with which the UV disinfection unit (1) is mounted on the inner side of a drum door (22) of the laundry treatment apparatus (2).

3. The UV disinfection unit (1′) according to claim 1, wherein the housing (10) is formed to be a freely movable in the drum (21), self-sustaining, three-dimensional object.

4. The UV disinfection unit (1, 1′) according to claim 1, wherein the sensor provided in the housing (10) is at least one of a positional sensor (13), a movement sensor (14) and a temperature sensor (18).

5. The UV disinfection unit (1, 1′) according to claim 1, wherein a germ sensor (17) is arranged in the housing (10).

6. The UV disinfection unit (1, 1′) according to claim 1, wherein the UV light source (11) comprises several UVC-emitting LED semiconductors (11) spatially distributed in the housing (10), which are arranged in the housing (10) with a UV light transparent dividing wall.

7. The UV disinfection unit (1, 1′) according to claim 1, wherein the UV disinfection unit (1, 1′) is adapted to being charged with a charging tray (32) for inductive charging of the electrical energy store (15), wherein the UV disinfection apparatus (1, 1′) is adapted to being placed on the charging tray (32) for charging.

8. The UV disinfection unit (1, 1′) according to claim 1, wherein the UV disinfection unit (1, 1′) is provided with a cover (31), wherein the cover (31) is adapted to being installed on the outer side of the drum door (22).

9. The UV disinfection unit (1, 1′) according to claim 1, wherein the sensor provided in the housing (10) is a positional sensor (13) wherein reference points are assigned complementary to the UV disinfection unit (1, 1′) which are attachable to the laundry treatment device (2) and cooperate with the position sensor (13).

10. A method for disinfecting laundry in a rotatable drum (21) of a laundry treatment machine with a self-sufficient UV disinfection unit (1, 1′) according to claim 1, comprising
placing the self-sufficient UV disinfection unit (1, 1′) with the laundry in the drum (21) of the laundry treatment machine, and
turning on the UV light source (11) in the UV disinfection unit (1, 1′) only upon operation of the laundry treatment machine.

11. The method of claim 10, wherein the UV disinfection unit (1, 1′) is switched on by means of sensors responsive to rotation movement of the drum (21) and turned off upon completion of the rotation movement.

12. A method according to claim 10, wherein a movement profile is provided stored in the sensor system for detecting the rotation movement of the drum (21).

13. The method of claim 10, wherein the quality of disinfection in the fluid circulation in the laundry treatment machine (2) is measured and dependent thereon at least one of the intensity and duration of the UV irradiation is controlled.

14. The method according to claim 10, wherein the laundry treatment machine is a washing machine (2) or in a dryer (2).

15. The method according to claim 14, wherein the washing machine (2) or dryer (2) includes a drum (21), and wherein the UV disinfection apparatus (1, 1′) is placed or set in the drum (21) of the washing machine (2) or of the dryer (2).

16. The UV disinfection unit (1′) according to claim 1, wherein the housing (10) is formed to be a freely movable in the drum (21), self-sustaining, three-dimensional object in the form of a ball.
